(19) United States
(12) Patent Application Publication
(10) Pub. No.: US 2007/0042040 A1
(43) Pub. Date: Feb. 22, 2007

(54) Methods and compositions using gonadotropin hormone releasing hormone

(76) Inventors: Herve Porchet, Cugy (CH); Frederic Heimgartner, Villeneuve (CH); Catherine Curdy, Martigny (CH); Bertrand Ducrey, Martigny (CH)

Correspondence Address: NIXON & VANDERHYE, PC, 901 NORTH GLEBE ROAD, 11TH FLOOR, ARLINGTON, VA, 22203, US

(21) Appl. No.: 10/554,292

(22) PCT Filed: Apr. 30, 2004
(86) PCT No.: PCT/IB04/01334

§ 371 Date: Sep. 22, 2006

(30) Foreign Application Priority Data
Apr. 30, 2003 (WO) PCT/IB03/01680

(51) Int. Cl. A61K 9/22; A61K 31/56; A61K 38/24

(52) U.S. Cl. 424/468; 514/8; 514/171

(57) ABSTRACT

The present invention relates to compositions comprising two sustained release formulations, the first being capable of releasing a gonadotropin releasing hormone composition and the second an estrogenic composition. The compositions of the invention can be employed for an improved androgen deprivation therapy of prostate cancer, in which therapy loss of bone mineral density and the occurrence and severity of hot flashes are minimized through the maintenance of a minimally adequate estrogen level.

### BACKGROUND AND PRIOR ART

Gonadotropin hormone releasing hormone (GnRH) agonists and antagonists have been used to treat benign gynaecological disorders including premenstrual syndrome and androgen-dependent cancer of the prostate. GnRH is also known as luteinizing hormone-releasing hormone. GnRH is secreted by the hypothalamus in the pituitary portal system in a pulsating fashion. Because the hormone has a half-life of the order of minutes, the pituitary gland is exposed to pulses of hormone. This exposure results in the secretion of the gonadotropins, i.e., luteinizing hormone (LH) and follicle-stimulating hormone (FSH). In men LH acts on the Leydig cells of the testes, stimulating the secretion of testosterone. FSH is responsible for spermatogenesis. Testosterone appears to feedback-inhibit secretion of GnRH and reduce the sensitivity of the pituitary to the hormone. In women FSH acts on the ovaries, stimulating secretion of estrogen. The main functions of LH in women are to support follicular maturation and to trigger ovulation at mid-follicular cycle. Like testosterone, estrogen appears to be capable of feedback inhibition of GnRH secretion and action.

Administration of potent agonists of GnRH was found to cause an initial flare-up of LH and FSH release that is followed by a complete down-regulation of GnRH receptor in the pituitary. As a consequence, LH and FSH are no longer released, and sex hormones are reduced to oophorectomized levels in women and orchiectomized or castrate levels in men, respectively. The development of high-dose depot formulations of GnRH agonists permitted sustained inhibition of sex steroid production and ease of drug administration.

Typically, prostate cancer is initially androgen-dependent and only in late stages becomes androgen-independent. Various methods of androgen ablation therapy were practiced, either as the only therapy or in conjunction with other treatment modalities such as surgery, external beam radiation therapy, brachytherapy, etc. An oral regimen of high doses of the semi-synthetic estrogenic compound diethylstilbesterol was one of the earliest non-surgical options for the treatment of prostate cancer. This therapy was equally effective in mediating remission as orchiectomy. Unfortunately, high doses of the estrogenic compound administered orally caused cardiovascular complications, including edema and deep vein thrombosis. Diethylstilbesterol therapy was abandoned when GnRH agonists and antagonists, which essentially lacked cardiovascular toxicity became available.

While GnRH agonists are clinically equally effective in inducing prostate cancer remission as orchiectomy, the gold standard of treatment, their use is accompanied by important other toxicities, including fatigue, weight gain, depression, bone loss, anaemia, muscle atrophy, gynecomastia, hot flashes, loss of cognitive function, and decrease in high-density lipoprotein. Hellerstedt and Pienta. CA Cancer J Clin 2002; 52: 154-179. Perhaps, the complications that most severely affect quality of life are loss of bone mineral density and hot flushes.

Because testosterone is the main circulating sex hormone in men it was long assumed that the increased bone turnover and loss of bone mineral density in chirurgically castrated men or in prostate cancer patients treated with GnRH agonists or antagonists was due to the absence of this hormone. However, recent observational studies suggested, surprisingly, that bone mineral density in men correlated better with estrogen levels than with testosterone levels. Khosla et al. J Clin Endocrinol Metab 2002; 87: 1443-1450. An interventional study showed that estrogen supplementation prevented the GnRH-induced reduction in bone formation markers as well as the increase in bone resorption markers in elderly men treated with a GnRH agonist. Khosla et al. J Clin Endocrinol Metab 2001; 86: 3555-3561. Finally, another study showed that specific inhibition of aromatase activity also resulted in a significant increase in bone resorption markers and a decrease in bone formation markers. Taxel et al. J Clin Endocrinol Metab 2002; 87:4907-4913.

### SUMMARY OF THE INVENTION

The invention relates to compositions comprising a first sustained release formulation of a gonadotropin hormone releasing hormone (abbreviated GnRH herein) composition capable of releasing the GnRH composition during a period of at least about one month, preferably at least two months and more preferably at least three months, at a rate sufficient to induce and maintain chemical castration of a male patient, and a second sustained release formulation of an estrogenic composition capable of maintaining for said period a serum level sufficient to reduce the enhanced loss of bone mineral density or the hot flashes that are normally caused by the administration of a GnRH composition that chemically castrates a male patient.

Preferably, the first sustained release formulation of a composition of the invention releases a GnRH composition at a rate of between about 10 and about 1,000 μg per day. The second sustained release formulation of the invention releases an estrogenic composition under a profile comprising at least a first initial phase and a second phase. In the course of the first initial phase, the second sustained release formulation of the invention displays an attenuated initial burst. In the course of the second phase, the second sustained release formulation releases the estrogenic composition at a rate between about 10 and 100 μg of estradiol equivalent per day, preferably at a rate not exceeding about 50 μg of estradiol equivalent per day. Preferably, the release of the estrogenic composition in the course of the first initial phase never exceeds 5 times, more preferably 3 times, the upper daily release of the estrogenic composition occurring during the second phase.

In a different embodiment of the invention the composition is not limited by reference to chemical castration of a male patient. It is defined as comprising a first sustained release formulation of a GnRH composition capable of releasing the GnRH composition for a period of at least about one month at an average rate between about 10 and 1,000 μg per day and a second sustained release formulation of an estrogenic composition capable of releasing during said period the estrogenic composition under a profile comprising at least a first initial phase and a second phase as defined above.

In the compositions of the invention the GnRH composition of the first sustained release formulation is selected from the group consisting of GnRH, agonists of GnRH, antagonists of GnRH and mixtures thereof. Preferably, the GnRH composition is a GnRH agonist selected from the group consisting of leuprorelin, goserelin, triptorelin, buserelin, nafarelin, deslorelin, histerelin, gonadorelin, and salts and mixtures thereof.

The estrogenic composition present in the second sustained release formulation is selected from the group consisting of chlorotrianisene, dienestrol, diethylstilbestrol, diethylstilbestrol dipropionate, diethylstilbestrol monobenzyl ether, equilelinin, equilelinin sulfate, estetrol, estradiol, (3α,17β)-estr-4-ene-3,17-diol, estriol, estriol hemisuccinate, estrone, estrone sulfate monosodique, estrone potassium sulfate, ethinylestradiol, fosfestrol tetrasodique, hexestrol, hydroxyestrone diacetate, mestranol, pinestrol, piperazine estrone sulfate, promestriene, quinestrol, tamoxifen, toremifene, raloxifene, lasofoxifene and mixtures thereof.

In preferred compositions the GnRH composition of the first sustained release formulation is triptorelin or a salt thereof, and the estrogenic composition of the second sustained release formulation is estradiol. In most preferred compositions the GnRH composition of the first sustained release formulation is triptorelin, or a salt thereof, that is released at a rate of about 100 μg per day, and the estrogenic composition of the second sustained release formulation is estradiol that is released at a rate of between about 25 and 50 μg per day in the course of said second phase.

The invention further relates to a method for the treatment of prostate cancer, involving administration to a prostate cancer patient of a composition comprising a first sustained release formulation of a GnRH composition capable of releasing the GnRH composition during a period of at least about one month, preferably at least two months and more preferably at least three months, at a rate sufficient to induce and maintain chemical castration of the patient, and a second sustained release formulation of an estrogenic composition capable of maintaining for said period a serum level sufficient to reduce the enhanced loss of bone mineral density or the hot flashes that are normally caused by the administration of a GnRH composition that chemically castrates a male patient.

Preferably, the first sustained release formulation of a composition administered to a prostate cancer patient releases a GnRH composition at a rate of between about 10 and about 1,000 μg per day, and the second sustained release formulation releases a estrogenic composition at a rate between about 10 and 100 μg per day. Most preferably, the second sustained release composition administered to a prostate cancer patient according to the method of the invention releases an estrogenic composition under a profile comprising at least a first initial phase, with an attenuated burst of release, and a second phase as described above.

In the compositions administered according to the method of the invention the GnRH composition of the first sustained release formulation is selected from the group consisting of GnRH, agonists of GnRH, antagonists of GnRH and mixtures thereof. Preferably, the GnRH composition is a GnRH agonist selected from the group consisting of leuprorelin, goserelin, triptorelin, buserelin, nafarelin, deslorelin, histerelin, gonadorelin, and salts and mixtures thereof.

The estrogenic composition present in the second sustained release formulation is selected from the group consisting of chlorotrianisene, dienestrol, diethylstilbestrol, diethylstilbestrol dipropionate, diethylstilbestrol monobenzyl ether, equilelinin, equilelinin sulfate, estetrol, estradiol, (3α, 17β)-estr-4-ene-3,17-diol, estriol, estriol hemisuccinate, estrone, estrone sulfate monosodique, estrone potassium sulfate, ethinylestradiol, fosfestrol tetrasodique, hexestrol, hydroxyestrone diacetate, mestranol, pinestrol, piperazine estrone sulfate, promestriene, quinestrol, tamoxifen, toremifene, raloxifene, lasofoxifene and mixtures thereof.

In preferred compositions administered according to the method of the invention the GnRH composition of the first sustained release formulation is triptorelin, or a salt thereof, and the estrogenic composition of the second sustained release formulation is estradiol. In most preferred compositions of the method of the invention the GnRH composition of the first sustained release formulation is triptorelin, or a salt thereof, that is released at a rate between about 100 μg per day, and the estrogenic composition of the second sustained release formulation is estradiol that is released at a rate of between about 25 and 50 μg per day. Compositions of the invention can be administered by a subcutaneous, intramuscular, or transdermal route.

### DETAILED DESCRIPTION OF THE INVENTION

The present invention relates to novel compositions and the use of these compositions to treat hormone-responsive prostate cancer without eliciting the severe side effects characteristic of prior art hormone ablation therapies. The compositions of the invention comprise two sustained release formulations, the first comprising a gonadotropin hormone releasing hormone (GnRH) composition and the second an estrogenic composition, that are administered to a patient simultaneously. The formulations may be combined at the time of administration or may be joined at the time of manufacture. Typically, the sustained release formulations of the invention are effective for a period of at least about one month. The period of effectiveness may be as long as one year. Formulations that are even longer-lasting are considered as being within the scope of the present invention. Preferably, the compositions of the invention are designed for treatment periods of one to three months, after which periods the compositions are re-administered.

The first sustained release formulation comprises a GnRH composition. A number of compounds were described that inhibit secretion of gonadotropins and, consequently, the secretion of androgens in men and estrogens in women. In men estrogens are derived from testosterone by the aromatase reaction. GnRH compositions include both agonists and antagonists of GnRH as well as GnRH itself. GnRH compositions of the invention may also consist of mixtures of the latter compounds. GnRH antagonists act by competing with GnRH for GnRH receptor in the pituitary gland. Normally, GnRH is secreted in a pulsating fashion. Because of the high turnover of the hormone, GnRH receptors are exposed to waves of GnRH signalling release of LH and FSH. In the presence of high concentrations of a GnRH agonist, after an initial bust of LH and FSH release, the signalling pathway is shut down through down-regulation of GnRH receptor and reduction of LH and FSH release. Within a period of several weeks, LH and FSH release are completely suppressed, and estrogen and testosterone concentrations reach oophorectomized levels in women and orchiectomized or castrate levels in men, respectively. In the presence of such minimal levels of testosterone and estrogen, feedback inhibition of GnRH no longer occurs. Consequently, GnRH release is maximal. This release pattern assists the maintenance of GnRH receptor down-modulation. Well-known GnRH agonists include leuprorelin, goserelin, triptorelin, buserelin, nafarelin, deslorelin, histrelin, gonadorelin and salts thereof. A well-known GnRH antagonist is abarelix.

A variety of sustained release formulations of GnRH agonists were developed and are commercially available. Examples of commercial sustained release formulations of GnRH agonists include Lupron Depot 3.75 mg and Lupron Depot 7.5 mg of TAP Pharmaceuticals Inc. of Lake Forrest, Ill. Lupron Depot 3.75 mg comprises 3.75 mg leuprorelin acetate, 0.65 mg gelatin, 33.1 mg DL-lactic and glycolic acids co-polymer, and 6.6 mg D-mannitol. The accompanying diluent contains 7.5 mg carboxymethylcellulose sodium, 75 mg mannitol, 1.5 mg polysorbate 80, water, USP, and glacial acetic acid. Lupron Depot —3 Month 22.5 mg is a formulation for intramuscular injection at three months intervals comprising 22.5 mg leuprorelin acetate in polylactide microspheres. U.S. Pat. Nos. 4,728,721; 4,849,228; 5,330,767; 5,476,663; 5,480,656; 5,575,987; 5,631,020; 5,643,607; 5,716,640; 5,814,342; 5,823,997; 5,980,488; 6,036,976. Other sustained release formulations of leuprorelin acetate include Eligard, a one-month formulation by Atrix Laboratories and Viadur, a 12-months formulation by ALZA Corporation. Zoladex 3.6 mg and 10.8 mg are one-month and three-months depot formulations, respectively, of goserelin acetate marketed by AstraZeneca. The Zoladex 3.6 mg formulation comprises goserelin acetate in an amount corresponding to 3.6 mg of goserelin in 13.3-14.3 mg D,L-lactic and glycolic acids co-polymer. Decapeptyl distributed by Ferring Corp. and Ipsen-Beaufour is a depot formulation of triptorelin acetate or pamoate. The one-month formulation of Decapetyl includes 3.75 mg triptorelin encapsulated in polylactide co-glycolide microcapsules. Similar sustained release formulations of triptorelin pamoate have been approved recently by the health authorities in Germany under the name Pamorelin. Pamorelin is available as one-month or three-months sustained release formulation (Pamorelin Depot 3.75 mg, Pamorelin LA 11.25 mg). Pamorelin Depot 3.75 mg is a sterile, lyophilised biodegradable microgranule formulation supplied as a single dose vial containing triptorelin pamoate (3.75 mg of triptorelin peptide), 170 mg poly-d,l-lactide-co-glycolide, 85 mg mannitol, 30 mg carboxymethylcellulose sodium and 2 mg polysorbate 80. For injection, the formulation is suspended in 2 ml water and injected intramuscularly. Pamorelin LA 11.25 mg is a similar formulation containing triptorelin pamoate (11.25 mg of triptorelin peptide), 145 mg poly-d,l-lactide-co-glycolide, 85 mg mannitol, 30 mg carboxymethylcellulose sodium and 2 mg polysorbate 80. The formulation is suspended in 2 ml water and injected intramuscularly. Similar formulations are described in U.S. Pat. Nos. 5,134,122, 5,192,741 and 5,225,205. These patents are incorporated herein in their entirety by this reference.

Analogous sustained release formulation of GnRH, a GnRH agonist, a GnRH antagonist or mixtures thereof can be used in the compositions of the invention. Such sustained release formulations may be based on biodegradable and/or biocompatible polymers other than the polylactide-glycolide co-polymers present in the above-described commercial formulations, including ethylene vinyl acetate, polyanhydrides, polyglycolic acid, collagen, polyorthoesters and polylactic acid. These and other polymers as well as methods for preparing appropriate formulations using such polymers are well known to those skilled in the art.

While the first sustained release formulation of the present invention is preferably a depot formulation of triptorelin pamoate such as the Pamorelin formulations, other sustained release formulations of an agonist or antagonist of GnRH, or of GnRH itself, could also be employed. Any depot formulation that continuously releases an agonist or antagonist of GnRH or GnRH at a rate sufficient to cause down-modulation of GnRH receptor and reduction of sex hormone concentrations to oophorectomized levels in women and orchiectomized or castrate levels in men would be suitable for use with the present invention. While the exact rate of release may vary with the nature of the GnRH agonist (including GnRH) or antagonist used, the nature of the formulation, and the mode of administration, a suitable first sustained release formulation will release a GnRH agonist or antagonist at a rate of between about 10 and 1,000 μg per day.

Release of agonist or antagonist of GnRH from a first sustained release formulation will produce the well-known side effects of GnRH agonist/antagonist therapy. To counteract these side effects, in particular loss on bone mineral density and hot flashes in prostate cancer patients, the compositions of this invention comprise a second sustained release formulation that releases an estrogenic composition. Observational studies indicate that loss of bone mineral density in men may not occur if the serum level of bioavailable estradiol is at or above about 11 pg/ml. Khosla et al. J Clin Endocrinol Metab 2002; 87:1443-1450. Taking into account the increased level of sex hormone binding globulin in elderly men, this corresponds to a total serum estradiol level of minimally about 30 pg/ml.

Estrogenic compositions delivered by the second sustained release formulations include both natural and synthetic compounds. The preferred estrogenic composition is estradiol (chemical name: β-estra-1,3,5(10)-triene-3,17-diol; CAS RN: 50-28-2). Examples of other estrogenic compositions that can be used according to the present invention include chlorotrianisene, dienestrol, diethylstilbestrol, diethylstilbestrol dipropionate, diethylstilbestrol monobenzyl ether, equilelinin, equilelinin sulfate, estetrol, estriol, estriol hemisuccinate, estrone, estrone sulfate monosodique, estrone potassium sulfate, ethinylestradiol, fosfestrol tetrasodique, hexestrol, hydroxyestrone diacetate, mestranol, pinestrol, piperazine estrone sulfate, promestriene, quinestrol, and mixtures thereof. Because the potencies and pharmacokinetic properties of these estrogenic compositions are widely different, amounts of such estrogenic compositions to be used and concentrations to be reached will vary widely. For the purposes of this invention, amounts and concentrations of estrogenic compositions are defined by their equivalence to amounts and concentrations of estradiol. Equivalence means similarity of desirable biological effects achieved, e.g., reduction in loss of bone mineral density and/or reduction in frequency and severity of hot flashes in prostate cancer patients undergoing hormone ablation therapy.

Additional estrogenic compositions include selective estrogen receptor modulators (SERM) such as tamoxifen, toremifene, raloxifene, tibolone and lasofoxifene. Riggs & Hartman. 2003. N Engl J Med 348, 618-629. Ke et al. 2001. J Bone Miner Res 16, 765-773. Because of the selectivity of these compositions, their use in a second sustained release formulation of this invention may only produce some but not all of the beneficial effects resulting from estradiol administration. For example, raloxifene, toremifene and tamoxifen can be expected to slow bone resorption but not to reduce (but, possibly, to enhance) the incidence and severity of hot flashes. Estrogenic compositions also include so-called ANGELS (Activators of Non-Genotropic Estrogen-like Signaling) compounds that were described in patent application PCT/US02/18544. ANGELS compounds are small molecules that mimic the non-genotropic effects of estrogen and androgen but substantially lack their genotropic effects. A preferred ANGELS compound is (3α,17β)-estr-4-ene-3,17-diol (CAS RN: 35950-87-9) that was shown to reverse bone loss in mouse models. Kousteni et al. 2002. Science 298, 843-846.

Estrogens are well known to increase the probability of cardiovascular events, in particular edema and deep venous thrombosis. This realization was an important reason why oral diethylstilbesterone therapy was abandoned for GnRH agonist therapies. Analogous observations were made for estrogen replacement therapies for postmenopausal women. Although the toxicity of estrogens to prostate cancer patients may be mitigated to some extent if the route of administration of the hormone is changed from oral to parenteral, there still may be a significant remainder risk associated with the administration of elevated doses of estrogens.

To effectively counteract the negative side effects of GnRH administration without unnecessarily increasing the risk associated with high levels of estrogens, the second sustained release formulation releases an estrogenic composition at a low rate that is calculated to be only sufficient to provide a serum estrogen level equivalent to about 30 pg/ml of estradiol. Because of biological differences between subjects, the actual serum estradiol or estradiol equivalent level achieved by administration of the second sustained release formulation may vary between about 10 pg/ml and about 50 pg/ml.

However, one of the drawbacks of sustained release formulations is that they almost inevitably show a bimodal kinetics of drug release, comprising an initial burst of release that is followed by a prolonged phase of sustained release at a considerably lower rate. Such a release profile would be dissuasive enough for contemplating the use of such formulations for the present purpose.

It has been surprisingly found that some of the second sustained release formulation of the composition of this invention display a release profile that approaches unimodality. This was obtained by selecting, for a given estrogenic composition, the right compromise between the biodegradable polymeric material in which such composition is embedded and the conditions on how to conduct the process for the preparation of the formulation. One of the polymeric materials which had demonstrated to offer the appropriate formulation was a poly(D,L-lactide-co-glycolide), preferably the one in which the ratio between respectively the two copolymers is comprised between 85:15 and 40:60, for instance 50:50 or 65:35. Preferably, the appropriate formulation is under the form of microspheres and one of the method for preparing the same may be the one known by specialist as emultion/solvant extraction. Optionally, mixing at least two sustained release formulations obtained from different batches may help to smooth down the release profile.

Accordingly, because of the absence of an important initial burst of drug release from this second sustained release formulation, estrogen concentrations will never greatly exceed target levels. The calculated ideal rate of release of estrogenic composition is equivalent to about 25 μg/day of estradiol (clearance x desired serum level or increase in serum level). The maximal rate of release of estrogenic composition during the first days following administration of the second slow release formulation will be equivalent to about 75 μg estradiol per day. As a consequence of these narrowly defined release characteristics of the second sustained release formulation, the risk associated with a high estrogen level will be kept to a minimum.

The two sustained release formulations of the composition of the present invention, the first comprising a gonadotropin hormone releasing hormone (GnRH) composition and the second an estrogenic composition, may be combined at the time of administration or may be joined at the time of manufacture. The separated or combined formulations may be stored as a solid form, for instance as a lyophilisat.

The composition of the present invention is administered as a single intramuscular injection, for instance in the buttock, after having re-constituted an injectable preparation.

Typically, the sustained release formulations of the invention are effective for a period of at least about one month, preferably at least two months, more preferably at least three months. Preferably, the compositions of the invention are designed for treatment periods of one to three months, after which periods the compositions are re-administered.

The composition of the present invention and its properties are presented in more details in the following examples and the drawing in which:

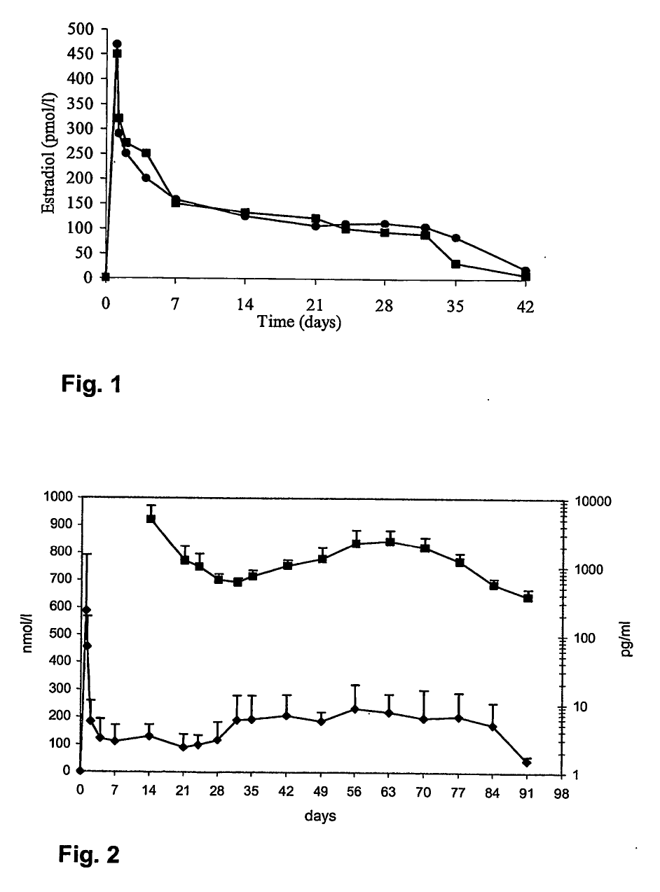

FIG. 1 represents estradiol kinetic profiles as obtained with formulation of Example 1 (square) and with formulation of Example 2 (circle);

FIG. 2 represents estradiol kinetic profile (lozenge) and triptorelin kinetic profile (square) as obtained with combined composition of Example 3; and

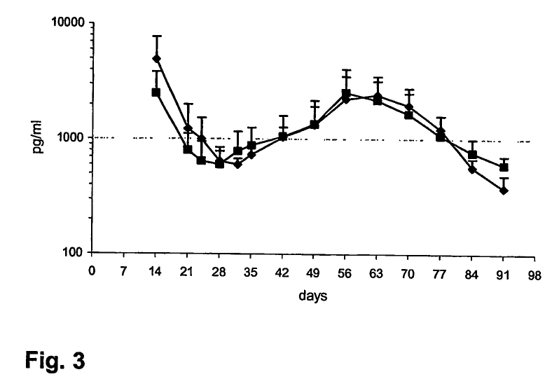

FIG. 3 represents triptorelin release profiles as obtained with the reference long lasting triptorelin formulation (square) and with the combined composition of Example 3 (lozenge).

### EXAMPLE 1

### Preparation of a Composition Releasing Triptorelin and Estradiol Over a Period of at Least One Month

1. Formulation of Triptorelin

This formulation was obtained according to the method described in U.S. Pat. No. 5,134,122, Example 1.

2. Formulation of Estradiol Embedded into PLGA Microspheres

An aqueous phase (Solution A) was prepared by mixing under magnetic agitation 160 g of PVA (polyvinyl alcohol) and 7840 g H₂O MilliQ at a temperature of 40° C. Next, an organic phase (Solution B) was prepared by dissolving 4.9 g of polymer 50:50 poly (D,L-lactide-co-glycolide) (inherent viscosity (iv)=0.42 dl/g) in 50 g of ethyl acetate under magnetic agitation. 100 mg of estradiol were dissolved in 800 μl of DMSO (solution C).

Solutions B and C were mixed together and the obtained solution was pumped into the homogenisation chamber at a rate of 5 ml/minute. Solution A was pumped in parallel at a rate of 750 ml/minute into the homogenisation chamber. The rotation speed of the rotor was 5000 rpm and the process lasted about 10 minutes.

The suspension thus obtained was filtered on 1.2 μm and the particles were then recuperated by filtration, washed with water, followed by lyophilization. The core loading is 1.50% and the mean size D(v,0.5) is 18.9 μm.

Estradiol serum release in rat following a single intramuscular injection of the obtained formulation is reported in Example 4.

### EXAMPLE 2

### Preparation of a Composition Releasing Triptorelin and Estradiol Over a Period of at least one month

1. Formulation of Triptorelin

This formulation was obtained according to the method described in U.S. Pat. No. 5,134,122, Example 1.

2. Formulation of Estradiol Embedded into PLGA Microspheres

An aqueous phase (Solution A) was prepared by mixing under magnetic agitation 80 g of PVA (polyvinyl alcohol) and 3920 g H₂O MilliQ at a temperature of 40° C. Next, the organic phase (Solution B) was prepared by dissolving 4.5 g of polymer poly 65:35 poly (D,L-lactide-co-glycolide) (inherent viscosity (iv)=0.62 dl/g) in 25 g of ethyl acetate under magnetic agitation. 92 mg of estradiol were dissolved in 800 μl of DMSO (solution C).

Solutions B and C were mixed and this solution was pumped into the homogenization chamber at a rate of 5 ml/minute. Solution A was pumped in parallel at a rate of 630 ml/minute into the homogenization chamber. The rotation speed of the rotor is 5000 rpm and the process lasted about 6 minutes.

The suspension thus obtained was filtered on 1.2 μm and the particles were then recuperated by filtration, washed with water, followed by lyophilization. The core loading is 1.60% and the mean size D(v,0.5) is 32.2 μm.

Estradiol serum release in rat following a single intramuscular injection of the obtained formulation is reported in Example 4.

### EXAMPLE 3

### Preparation of a Composition Releasing Triptorelin and Estradiol Over a Period of at Least Three Months

1. Formulation of Triptorelin

A formulation of microgranules of triptoreline pamoate was prepared according to the following method.

Approximately 12 wt % of triptoreline pamoate was mixed with approximately 88 wt % PLGA 75:25 in a ball mill, at room temperature. The given mixture was duly homogenized, subjected to a progressive compression and simultaneously to a progressive heating, before extruded at a temperature of approximately 110° C. The extrudate was cut into pellets and ground at a temperature of about −100° C. The microgranules obtained after grinding were sieved below 180 microns.

2. Formulation of Estradiol

A formulation of microspheres of estradiol and PLGA 50/50 having an inherent viscosity of 0.42 dL/g (formulation 1) was prepared as follows:

The aqueous phase (Solution A) was prepared by mixing under magnetic agitation 160 g of PVA (polyvinyl alcohol) and 7840 g H₂O MilliQ at a temperature of 40° C. Next, the organic phase (Solution B) was prepared by dissolving 4.9 g of polymer 50:50 poly (D,L-lactide-co-glycolide) (inherent viscosity (iv)=0.42 dl/g) in 50 g of ethyl acetate under magnetic agitation. 100 mg of estradiol were dissolved in 800 μl of DMSO (solution C).

Solutions B and C were mixed and this solution was pumped into the homogenization chamber at a rate of 5 ml/minute. Solution A was pumped in parallel at a rate of 750 ml/minute into the homogenization chamber. The rotation speed of the rotor was 5000 rpm and the process lasted about 10 minutes.

The suspension thus obtained was filtered on 1.2 μm and the particles were then recuperated by filtration, washed with water, followed by lyophilization. The core loading is 1.50% and the mean size D(v,0.5) is 18.9 μm.

A formulation of microspheres of estradiol and PLGA 85/15 having an inherent viscosity of 0.6 dL/g (formulation 2) was prepared as follows:

The aqueous phase (Solution A) was prepared by mixing under magnetic agitation 160 g of PVA (polyvinyl alcohol) and 7840 g H₂O MilliQ at a temperature of 40° C. Next, the organic phase (Solution B) was prepared by dissolving 4.65 g of polymer 85:15 poly (D,L-lactide-co-glycolide) (inherent viscosity (iv)=0.6 dl/g) in 50 g of ethyl acetate under magnetic agitation. 350 mg of estradiol were dissolved in 2500 μl of DMSO (solution C).

Solutions B and C were mixed and this solution was pumped into the homogenization chamber at a rate of 5 ml/minute. Solution A was pumped in parallel at a rate of 750 ml/minute into the homogenization chamber. The rotation speed of the rotor was 5500 rpm and the process lasted about 10 minutes.

The suspension thus obtained was filtered on 1.2 μm and the particles were then recuperated by filtration, washed with purified water, followed by lyophilization.

The core loading is 6.04% and the mean size D(v,0.5) is 18.4 μm.

3. Combined Formulation of Triptorelin and Estradiol

These formulations were mixed in a vial in order to have a 75:25 ratio of estradiol microspheres formulation 1 and 2 respectively, an estradiol dose of 3 mg and a triptoreline dose of 12 mg. The mixture was finally lyophilized (after addition of a lyophilization medium containing mannitol, sodium carboxymethylcellulose and Tween 80).

Estradiol and triptorelin serum releases in rat following a single intramuscular injection of the obtained formulation are reported in Example 4.

### EXAMPLE 4

### Phamacokinetics Studies

The aim of this experimental study was to follow the estradiol and/or triptorelin serum release following a single intramuscular injection of estradiol/triptorelin formulations in the rat.

1. Animals and Administration of Formulations

Under mild ether anaesthesia, male Sprague Dawley orchidectomized rats were given an intramuscular injection (i.m.) of estradiol and or triptorelin formulation, in the posterior thigh muscle. Six animals were studied per group. The day before the injection of the formulation (Day 0), a reference blood sample was collected. Each injection (estradiol dose ranged from 0.75 to 2.25 mg/kg and/Or triptorelin dose of 9 mg/kg) was carried out on Day 1 at time T0. This was considered as the reference time for the following blood samples.

2. Blood Sampling

Two blood samples were then collected on Day 1, the first one 1 hour after injection (T0+1h00) and the second one 6 hours after injection (T0+6h00). On the following days, i.e. from Day 2 to Day 42, blood samples was collected at the same time as that chosen for T0. Blood samples were collected in all groups until day 42. For the animals treated with the three-month formulation, additional weekly blood samples were taken until Day 91. At each time point, approximately 1.5 ml of blood were collected from the retro-orbital sinus (right or left eye) using hematocrit capillaries

3. Assays

Serum estradiol and/or triptorelin were measured in the serum of treated animals by Radio-Immuno-Assay (RIA).

4. Results

4.1 Estradiol Formulations as Obtained in Example 1 and Example 2

FIG. 1 reports the kinetic profile of the estradiol release of formulation of Example 1 (square) and of Example 2 (circle) in rat serum.

This profile shows a burst at 450 pmol/l and 470 pmol/l corresponding to the formulation of Example 1 and Example 2, respectively, whereas the plateau was at around 100 pmoles/l for both formulations. A ratio of 4.5-4.7 was obtained. After the burst, the estradiol level in serum decreased up to reach a plateau from day 7to day 32. Then, from day 32, the estradiol release decreased.

A similar kinetic profile is expected to be achieved in man.

4.2 Combined Formulation of Triptorelin and Estradiol as Obtained in Example 3

FIG. 2 reports the kinetic profile of estradiol (lozenge) and triptorelin (square) releases of combined formulation of Example 3 in rat serum during 84 days following a single intramuscular injection of formulation.

A serum estradiol burst at 588 pmol/l was observed just after the injection of the formulation. Then the estradiol level decreased rapidly to reach a plateau (between 90 and 130 pmol/l) from Day 7 until Day 28. From Day 35, a small increase in estradiol levels was observed (range from 190 to 234 pmol/l at Day 56) followed by a decrease from Day 84. A level of 45 pmol/l was still observed at Day 91. These results showed that the formulation can induce a regular release of estradiol in the serum, with a relatively initial burst.

FIG. 3 reports the compared triptorelin profiles of serum triptorelin release in rat serum following the IM injection of reference triptorelin formulation (triptorelin alone) (square) and of the combined triptorelin and estradiol formulation (lozenge) of Example 3.

The combination of estradiol and triptorelin did not modify the release of serum triptorelin compared to the three-month triptorelin formulation, as the two triptorelin serum profiles were similar.

### EXAMPLE 5

### Clinical Trial

A study comparing the effects of treatments respectively on bone mineral density, hot flushes, testosterone serum levels and prostate specific antigen in men receiving GnRH agonist therapy for prostate cancer by administrating a sustained release triptorelin (alone) and the combined triptorelin+estradiol formulation of Example 3.

140 men suffering from advanced prostate cancer without bone metastases are randomized to receive every 12 weeks injections of either a sustained release formulation of triptorelin pamoate 11.25 mg alone (reference) or triptorelin pamoate 11.25 mg combined with a dose of 3 mg of estradiol (composition of Example 3), both treatments intramuscularly in a sustained release (PLGA) formulation, 70 patients per treatment arm. The patients are followed over a 48-week period.

All patients are started on calcium and vitamin D supplements one month before start of the study drug treatment in order to prevent bone loss due to calcium or vitamin D insufficiency.

The bone mineral density (BMD) is measured at the baseline and at 48 weeks. The incidence and severity of hot flushes are measured at the baseline and monthly using a patient diary. The serum testosterone and prostate specific antigen (PSA) levels are measured at the baseline and at regular intervals.

Results:

Bone Mineral Density

At 48 weeks there is a 2.8% decrease in BMD at lumbar spine and 3.3% decrease in total hip in the triptorelin alone arm, whereas there is only a 0.5% decrease in BMD at both sites with triptorelin+estradiol treatment.

The mean difference in bone loss after 48 weeks between triptorelin+estradiol and triptorelin alone groups is found as 2.3% at lumbar spine and 2.8% at total hip (statistically significant), with a standard deviation of 4.4 in the change from baseline.

Hot Flushes

75% of the patients in both treatment arms experienced hot flushes. The mean number of hot flushes daily is 7 in the triptorelin alone group and 5 in the triptorelin+estradiol arm. The mean severity of hot flushes based on a visual analog scale (from 1 to 10) is 6.5 in the triptorelin alone arm and 4.5 in the triptorelin+estradiol arm.

Serum Testosterone Levels

The mean percentage of patients achieving castration (serum testosterone ≦1.735 nmol/L) at day 29 is 95.3% in the triptorelin alone arm, and 96.1% in the triptorelin+estradiol group. The mean percentage of patients maintaining castration between day 29 and day 336 is 98.2% in the triptorelin alone group and 98.5% in the triptorelin+estradiol group. The mean differences between the treatment groups are not significant.

Serum PSA Levels

The mean PSA concentrations decreased from 46.8 μg/L at baseline to 1.3 μg/L in the triptorelin alone group, and from 45.0 μg/L at baseline to 1.2 μg/L in the triptorelin+estradiol group. The mean differences between the treatment groups are not significant.

1. A composition consisting of:
a first sustained release formulation of a gonadotropin hormone releasing hormone composition capable of releasing the gonadotropin hormone releasing hormone composition during a period of at least about one month at a rate sufficient to induce and maintain chemical castration of a male patient, and
a second sustained release formulation of an estrogenic composition capable of maintaining for said period a serum level sufficient to reduce the enhanced loss of bone mineral density or the hot flashes that are normally caused by the administration of a gonadotropin hormone releasing hormone composition that chemically castrates a male patient,
characterised in that said second sustained release formulation releases an estrogenic composition under a profile comprising at least a first initial phase and a second phase, the release of the estrogenic composition in the course of said second phase being at a rate between about 10 and 100 mg of estradiol equivalent per day, the release of the estrogenic composition in the course of said first initial phase never exceeding 5 times the daily release of the estrogenic composition occurring during said second phase.

2. The composition according to claim 1, characterised in that said first sustained release formulation of a gonadotropin hormone releasing hormone composition is capable of releasing the gonadotropin hormone releasing hormone composition at a rate between about 10 and about 1,000 μg per day.

3. A composition consisting:
a first sustained release formulation of a gonadotropin hormone releasing hormone composition capable of releasing the gonadotropin hormone releasing hormone composition for a period of at least about one month at an average rate between about 10 and 1,000 μg per day, and
a second sustained release formulation of an estrogenic composition capable of releasing the estrogenic composition under a profile comprising at least a first initial phase and a second phase, the release of the estrogenic composition in the course of said second phase being at a rate between about 10 and 100 mg of estradiol equivalent per day, the release of the estrogenic composition in the course of said first initial phase never exceeding 5 times the upper daily release of the estrogenic composition occurring during said second phase.

4. The composition according to claim 1, characterised in that the gonadotropin hormone releasing hormone composition is selected from the group consisting of gonadotropin hormone releasing hormone, agonists of gonadotropin hormone releasing hormone, antagonists of gonadotropin hormone releasing hormone and mixtures thereof.

5. The composition according to claim 1, characterised in that the gonadotropin hormone releasing hormone composition is a gonadotropin hormone releasing hormone agonist selected from the group consisting of leuprorelin, goserelin, triptorelin, buserelin, nafarelin, deslorelin, histerelin, gonadorelin, and salts and mixtures thereof.

6. The composition according to claim 1, characterised in that the estrogenic composition is selected from the group consisting of chlorotrianisene, dienestrol, diethylstilbestrol, diethylstilbestrol dipropionate, diethylstilbestrol monobenzyl ether, equilelinin, equilelinin sulfate, estetrol, estradiol, (3α,17β)-estr-4-ene-3,17-diol, estriol, estriol hemisuccinate, estrone, estrone sulfate monosodique, estrone potassium sulfate, ethinylestradiol, fosfestrol tetrasodique, hexestrol, hydroxyestrone diacetate, mestranol, pinestrol, piperazine estrone sulfate, promestriene, quinestrol, tamoxifen, toremifene, raloxifene, lasofoxifene and mixtures thereof.

7. The composition according to claim 1, characterised in that the gonadotropin releasing hormone composition is triptorelin or a salt thereof and the estrogenic composition is estradiol.

8. The composition of claim 7, characterised in that triptorelin, or a triptorelin salt, is released at a rate of about 100 μg per day and estradiol is released at a rate between about 25 and 50 μg per day.

9. A method for the treatment of prostate cancer comprising:
administering to a patient suffering from prostate cancer a sustained release formulation of a gonadotropin hormone releasing hormone composition capable of releasing the gonadotropin hormone releasing hormone composition for a period of at least about one month at a rate sufficient to induce and maintain chemical castration of the patient, and
simultaneously administering to the patient a sustained release formulation of an estrogenic composition capable of maintaining for said period a serum level sufficient to reduce the enhanced loss of bone mineral density or the hot flashes that are normally caused by the administration of a gonadotropin hormone releasing hormone composition that chemically castrates a male patient.

10. A method for the treatment of prostate cancer comprising:
administering to a patient suffering from prostate cancer a sustained release formulation of a gonadotropin hormone releasing hormone composition capable of releasing the gonadotropin hormone releasing hormone composition for a period of at least about one month at an average rate between about 10 and 1,000 μg per day, and
simultaneously administering to the patient a sustained release formulation of an estrogenic composition under a profile comprising at least a first initial phase and a second phase, the release of the estrogenic composition in the course of said second phase being at a rate between about 10 and 100 mg of estradiol equivalent per day, the release of the estrogenic composition in the course of said first initial phase never exceeding 5 times the upper daily release of the estrogenic composition occurring during said second phase.

11. A method for the treatment of prostate cancer comprising:
administering to a patient suffering from prostate cancer a sustained release formulation of a gonadotropin hormone releasing hormone composition capable of releasing the gonadotropin hormone releasing hormone composition for a period of at least about one month at an average rate between about 10 and 1,000 μg per day, and
simultaneously administering to the patient a sustained release formulation of an estrogenic composition under a profile comprising at least a first initial phase and a second phase, the release of the estrogenic composition in the course of said second phase being at a rate between about 10 and 100 mg of estradiol equivalent per day, the release of the estrogenic composition in the course of said first initial phase never exceeding 5 times the upper daily release of the estrogenic composition occurring during said second phase.

12. A method as in claim 9, wherein the gonadotropin hormone releasing hormone composition is selected from the group consisting of gonadotropin hormone releasing hormone, agonists of gonadotropin hormone releasing hormone, antagonists of gonadotropin hormone releasing hormone and mixtures thereof.

13. A method as in claim 9, wherein the gonadotropin hormone releasing hormone composition is a gonadotropin hormone releasing hormone agonist selected from the group consisting leuprorelin, goserelin, triptorelin, buserelin, nafarelin, deslorelin, histerelin, gonadorelin, and salts and mixtures thereof.

14. A method as in claim 9, wherein the estrogenic composition is selected from the group consisting of chlorotrianisene, dienestrol, diethylstilbestrol, diethylstilbestrol dipropionate, diethylstilbestrol monobenzyl ether, equilelinin, equilelinin sulfate, estetrol, estradiol, (3a,17b)-estr-4-ene-3,17-diol, estriol, estriol hemisuccinate, estrone, estrone sulfate monosodique, estrone potassium sulfate, ethinylestradiol, fosfestrol tetrasodique, hexestrol, hydroxyestrone diacetate, mestranol, pinestrol, piperazine estrone sulfate, promestriene, quinestrol, tamoxifen, toremifene, raloxifene, lasofoxifene and mixtures thereof.

15. A method as in claim 9, wherein the gonadotropin releasing hormone composition is triptorelin or a salt thereof and the estrogenic composition is estradiol.

16. A method according to claim 15, wherein triptorelin, or a triptorelin salt, is released at a rate of about 100 μg per day and estradiol is released at a rate between about 25 and 50 μg per day.

17. A method as in claim 9, wherein the composition is administered by a subcutaneous, intramuscular, or transdermal route.

18. Use of a composition comprising:
a first sustained release formulation of a gonadotropin hormone releasing hormone composition capable of releasing the gonadotropin hormone releasing hormone composition during a period of at least about one month at a rate sufficient to induce and maintain chemical castration of a male patient, and
a second sustained release formulation of an estrogenic composition capable of maintaining for said period a serum level sufficient to reduce the enhanced loss of bone mineral density or the hot flashes that are normally caused by the administration of a gonadotropin hormone releasing hormone composition that chemically castrates a male patient, said serum level in estradiol equivalent being less than about 50 pg/ml.

19. Use of a composition comprising:
a first sustained release formulation of a gonadotropin hormone releasing hormone composition capable of releasing the gonadotropin hormone releasing hormone composition for a period of at least about one month at an average rate between about 10 and 1,000 μg per day, and
a second sustained release formulation of an estrogenic composition capable of releasing the estrogenic composition under a profile comprising at least a first initial phase and a second phase, the release of the estrogenic composition in the course of said second phase being at a rate between about 10 and 100 mg of estradiol equivalent per day, the release of the estrogenic composition in the course of said first initial phase never exceeding 5 times the upper daily release of the estrogenic composition occurring during said second phase,
for the preparation of a medicament for treatment of prostate cancer in a patient suffering from prostate cancer, said first sustained release formulation and said second sustained release formulation being simultaneously administrated to said patient.

20. The use according to claim 18, characterised in that the gonadotropin hormone releasing hormone composition is selected from the group consisting of gonadotropin hormone releasing hormone, agonists of gonadotropin hormone releasing hormone, antagonists of gonadotropin hormone releasing hormone and mixtures thereof.

21. The use according to claim 18, characterised in that the gonadotropin hormone releasing hormone composition is a gonadotropin hormone releasing hormone agonist selected from the group consisting leuprorelin, goserelin, triptorelin, buserelin, nafarelin, deslorelin, histerelin, gonadorelin, and salts and mixtures thereof.

22. The use according to claim 18, characterised in that the estrogenic composition is selected from the group consisting of chlorotrianisene, dienestrol, diethylstilbestrol, diethylstilbestrol dipropionate, diethylstilbestrol monobenzyl ether, equilelinin, equilelinin sulfate, estetrol, estradiol, (3a,17b)-estr-4-ene-3,17-diol, estriol, estriol hemisuccinate, estrone, estrone sulfate monosodique, estrone potassium sulfate, ethinylestradiol, fosfestrol tetrasodique, hexestrol, hydroxyestrone diacetate, mestranol, pinestrol, piperazine estrone sulfate, promestriene, quinestrol, tamoxifen, toremifene, raloxifene, lasofoxifene and mixtures thereof.

23. The use according to claim 18, wherein the gonadotropin releasing hormone composition is triptorelin or a salt thereof and the estrogenic composition is estradiol.

24. The use according to claim 23, wherein triptorelin, or a triptorelin salt, is released at a rate of about 100 μg per day and estradiol is released at a rate between about 25 and 50 μg per day.

25. The use according to claim 18, wherein the composition is administered by a subcutaneous, intramuscular, or transdermal route.
